(19) United States
(12) Patent Application Publication
(10) Pub. No.: US 2016/0263018 A1
(43) Pub. Date: Sep. 15, 2016

(54) Method For Treating Nicotine Withdrawal Symptoms

(71) Applicant: Trinity Pharma Group, New Port Richey, FL (US)

(72) Inventors: Kruitka Patel, Lutz, FL (US); Devan Patel, Tampa, FL (US)

(73) Assignee: Trinity Pharma Group, New Port Richey, FL (US)

(21) Appl. No.: 14/645,465

(22) Filed: Mar. 12, 2015

(51) Int. Cl. A61K 9/00; A61K 31/137; A61K 31/357; A61K 31/138; A61K 31/135; A61K 31/55

(52) U.S. Cl. CPC A61K 9/0014; A61K 31/135; A61K 31/55; A61K 31/357; A61K 31/138; A61K 31/137

(57) ABSTRACT

The present invention relates to a method for treating symptoms from a nicotine withdrawal comprising administering to a patient an effective amount of a transdermal compound. In one embodiment the compound includes the following active ingredients: Sertraline, Imipramine, Topiramate, and Bupropion, while in another embodiment the active ingredients are: Fluoxetine, Nortriptyline, Topiramate, and Bupropion.

### BACKGROUND OF THE INVENTION

The present invention is directed to the use of a new topical combination therapy for treating nicotine withdrawal symptoms.

Withdrawal symptoms from the absence of nicotine make quitting smoking very difficult and may include symptoms of depressed mood, insomnia, irritability, combativeness, frustration, anxiety, lack of concentration, restlessness, decreased heart rate and increased appetite resulting in weight gain. Studies show that the success rate 6 months after quitting smoking is higher in patients using some kind of nicotine replacement therapy (NRT) or combination of NRT and approved prescribed smoking cessation medication when compared to quitting abruptly. Using nicotine replacement products and/or medicine increases the chance of quitting.

FDA approved smoking cessation aids include bupropion (Zyban) and varenicline (Chantix) as first line agents and clonidine (Catapres) and nortriptyline (Pamelor) as second line agents with numerous other anti-depressants and pain modulators as off-label treatments in attempts to reduce the withdrawal effects.

Medication adherence has always been an uphill battle when treating smoking cessation patients for many different reasons. One of the more common causes of non-adherence to treatment is the side effects associated with traditional smoking cessation products, most notably the oral prescription products. Non-Adherence may also result from patients not fully understanding dosing and titration methods along with product's complicated directions for use and route of administration. Common limitations seen regarding nicotine replacement therapy are GI side effects, mouth soreness (lozenges and gum), hiccups, jaw aches (lozenges and gum), insomnia, and skin irritation (patches). Common side effects observed with prescription treatment options include increased heart rate, nausea and vomiting, headache, constipation, tremors and seizures, insomnia, flatulence and many others. The risk for drug interaction when taking these medications is also a consideration many patients will have in regards to their medical and psychiatric co-morbidities.

A need therefore exists to provide a product that when used can assist in managing the symptoms associated with nicotine withdrawal.

### SUMMARY OF THE INVENTION

The present invention is directed to a transdermal smoking cessation cream. In one embodiment there is provided a transdermal cream with active ingredients including: (a) Sertraline; (b) Imipramine; (c) Topiramate; and (d) Bupropion. In a second embodiment the active ingredients include: (a) Fluoxetine; (b) Nortriptyline; (c) Topiramate; and (d) Bupropion.

With this new multi-modal topical combination therapy combining TCAs, SSRIs, and atypical agents, several pathways dealing with the direct effects of the absence of nicotine via modulating nicotinic acetylcholine receptors as well as other neurotransmitters like serotonin, dopamine and norepinephrine will help prevent withdrawal symptoms.

### DETAILED DESCRIPTION OF THE INVENTION

The present invention is directed to a transdermal smoking cessation cream, helping smokers stop by suppressing nicotine withdrawal symptoms. This cutting edge cream formulation permits for multiple active ingredients combined into one simple formula without the unwanted side effects of current medication therapies. Clinical pharmacists specially formulate this custom compound medication. The topical therapy is non-invasive, in the form of a cream applied at the back of the neck at the hairline (referred to herein as the “BONATH region”), and provides a therapeutic dose quickly and effectively relieving nicotine withdrawal symptoms.

As known in the art, transdermal creams utilize the skin or mucosa to facilitate a rapid absorption of medications within the body. This is a very useful option for patients who may be unable to take medication orally or by injection. With transdermal absorption, the cream allows for medications to by-pass major organs and sidestep many common side effects associated with oral or injection medications. Transdermal creams also allow for the use of multiple medications in one single formula. This is helpful for patients who require multiple medications each day to treat an ailment. Topically applied drug need only traverse the stratum corneum of the skin to reach cutaneous free nerve endings for therapeutic effect. In contrast the transdermal patch requires a drug concentration gradient for active drug to enter blood vessels in the subcutaneous tissue and dermis, which in turn increases side effects.

In one embodiment there is provided a transdermal cream having a base cream, such as a Stera Base, with active ingredients to help moderate to severe withdrawal symptoms. While a specific base cream is discussed, the base cream may be substitute for a different cream as desired and without changing the scope of the invention. The cream is formulated to include the following: (a) Sertraline, with an active amount being in the range of 0.5% to 6%, and more preferably about 3%; (b) Imipramine, with an active amount being in the range of 0.1% to 5%, and more preferably about 0.5%; (c) Topiramate, with an active amount being in the range of 0.5% to 10%, and more preferably about 5%; and (d) Bupropion, with an active amount being in the range of 0.5% to 10%, and more preferably about 3%.

In a second embodiment, there is provided a transdermal cream having in a base cream, such as Versparo, with active ingredients to help mild withdrawal symptoms. While a specific base cream is discussed, the base cream may be substitute for a different cream as desired and without changing the scope of the invention. The cream is formulated to include the following: (a) Fluoxetine, with an active amount being in the range of 0.1% to 5%, and more preferably about 1%; (b) Nortriptyline, with an active amount being in the range of 0.1% to 5%, and more preferably about 1%; (c) Topiramate, with an active amount being in the range of 0.5% to 10%, and more preferably about 5%; and (d) Bupropion, with an active amount being in the range of 0.5% to 10%, and more preferably about 3%.

In either embodiment it is within the scope to use different bases or switching the bases such as incorporating Versparo in the first embodiment and Stera Base in the second embodiment.

SSRIs (Fluoxetine or Sertraline are referred to as SSRIs and are an antidepressant) are used to treat various types of anxiety and depression; two common symptoms of nicotine withdrawal. Topical SSRIs have been added to the formula to help manage withdrawal symptoms. Using SSRI's in combination with other ingredients that help smoking cessation will work synergistically together to help patients quit smoking.

Sertraline is a selective serotonin reuptake inhibitor (SSRI) that has indications in treating conditions of both Major Depressive Disorder (MDD) and General Anxiety Disorder (GAD). The main mechanism of action is to increase the amount of serotonin in the synaptic cleft by down-regulating the receptors responsible for reabsorption on the presynaptic neuron. Serotonin has been identified as having control in maintaining levels of motivation while deterring depression, anxiety and irritability that may be seen in patients suffering from withdrawals from quitting tobacco products. Sertraline is also proposed to have a mild effect on dopamine and norepinephrine pathways that will boost feelings of interest, satisfaction and feelings of well-being. Fluoxetine is categorized as a SSRI which, like sertraline, can be used in the treatment of both MDD and GAD. Fluoxetine has an excellent safety profile and again will help the patient with boosting motivation and decreasing depression, anxiety, and irritability in patients going through nicotine withdrawals.

Nortriptyline or Imipramine are Tricyclic Antidepressants, (TCA), used to enhance abstinence by decreasing withdrawal symptoms including irritability, food craving, concentration, restlessness and headache, all associated with nicotine withdrawal.

Nortriptyline is a tricyclic antidepressant with potent activity in increasing norepinephrine levels as well as increasing serotonin and dopamine. Nortriptyline is the most studied of the TCAs with respect to smoking cessation and has proven clinical efficacy in reducing cravings and increasing moods. It is a second line treatment because of the side effects associated with the product compared to the first line agents, however with topical application we are hoping to eliminate these side effects. In comparison with other TCAs, nortriptyline also has a low risk for developing hypotension and minimal reports of weight gain that are commonly associated with anti-cholinergic agents. Imipramine has a modality of increasing levels of norepinephrine, serotonin, and dopamine. Clinical effects seen with the administration of imipramine include maintaining a clear thought process, increasing levels of interest and motivation. Imipramine has limited studies in smoking cessation but has the same mechanism of action as nortriptyline. Results for smoking cessation are expected to be similar. Imipramine has less drowsiness associated with treatment versus nortriptyline.

Bupropion has been proven to help in smoking cessation and curb nicotine cravings, it may also help with other nicotine withdrawal symptoms including: depression, difficulty concentrating and weight gain. Oral Bupropion has been studied in a large number of clinical trials and is a first line option that is approved by the FDA for smoking cessation. Categorized as an atypical antidepressant, it works by increasing both dopamine and norepinephrine levels in the brain by inhibiting neuronal reuptake. These increased levels of dopamine are thought to be the main cause of aid in smoking cessation by reducing urges to smoke. Several clinical studies show significant efficacy in bupropion administration with long term smoking cessation success rates.

Topiramate is traditionally used as an anticonvulsant, and may aid in maintaining long-term smoking cessation and lessen nicotine cravings. Topiramate proves clinically beneficial in the treatment of anxiety and irritability seen during nicotine withdrawal by increasing the effects of GABA, the primary inhibitory neurotransmitter in the central nervous system. Synergistic mechanisms of actions also include the limiting of glutamate activity and blocking the spread of excitatory signaling via sodium channels, again reducing anxiety and irritability. This class of anti-depressants also exhibits anticholinergic activity that plays a pivotal part in weaning the patient off of the nicotinic acetylcholine receptor substrate, nicotine. A common side effect of topiramate is mild anorexia, something that may also prove useful to the patient in combating weight gain due to increased appetite during the smoking cessation process.

While noted above that the bases may be swapped or replaced with other bases, the following provides background on the two aforementioned bases Versapro and Stera Base. Versapro is an oil-free, natural emulsifying cosmetic cream used as a base in administering this multitude of topical agents. It is hypoallergenic, stable from pH 2 to 12, stable up to 45° C./113° F., and tolerant of salts to prevent pilling on the skin. All of these properties are needed as the product will be applied to the back of the neck up to 3-4 times daily for up to three months. Proper absorption of the medications and tolerability at the application site promotes optimal patient benefit and compliance.

Stera Base is a paraben free, oil-in-water emollient base with pH stability from 2-12 for use in the preparation of topical pharmaceutical applications. It acts as an all-day moisturizing cream with a non-greasy formula providing an irritant-free carrier for both lipophilic and hydrophilic medications. It has passed the USP microbial challenge test and provides excellent tolerance to active pharmaceutical ingredient base and salt forms.

As provided herein the above embodiments and active ingredients may have suitable replacement or additional ingredients. For example, clomipramine, desipramine, amitriptyline, doxepin, paroxetine, citalopram, niacin, nicotine, and/or clonidine may be combined in the treating of the withdrawal symptoms.

When compared to other smoking cessation treatments the above embodiments were found to decrease nausea, depression/anxiety, skin irritations, tremors/seizures, headaches, drowsiness, constipation, hiccups, insomnia/vivid dreams, mouth soreness/jaw ache, flatulence, and GI side effects with an increase of patient compliance with the treatment and handling of the withdrawal symptoms.

As noted above the embodiments should be applied to the back of the neck as needed for cravings and withdrawal symptoms. This topical administration will provide a more rapid clinical effect to the “pleasure center” of the brain in the limbic system. This topical administration technique targets the trigeminal nerve complex (TNC) by applying medication to the back of neck at the hair line.

From the foregoing and as mentioned above, it will be observed that numerous variations and modifications may be effected without departing from the spirit and scope of the novel concept of the invention. It is to be understood that no limitation with respect to the specific embodiments illustrated herein is intended or should be inferred. It is, of course, intended to cover by the appended claims all such modifications as fall within the scope of the claims.

1. A method for treating symptoms from nicotine withdrawal comprising administering to a patient an effective amount of a transdermal composition having the following ingredients: Sertraline, Imipramine, Topiramate, and Bupropion.

2. The method of claim 1, wherein the active ingredient of Sertraline has an active amount in the range of 0.5% to 6%.

3. The method of claim 1, wherein the active ingredient of Imipramine has an active amount in the range of 0.1% to 5%.

4. The method of claim 1, wherein the active ingredient of Topiramate has an active amount in the range of 0.5% to 10%.

5. The method of claim 1, wherein the active ingredient of Bupropion has an active amount in the range of 0.5% to 10%.

6. The method of claim 1, wherein the active ingredients have active amounts in accordance with the following: Sertraline, of 3%, Imipramine, of 0.5%; Topiramate, of 5%; and Bupropion, of 3%.

7. The method of claim 6, wherein the transdermal composition is placed in a base cream of Strera Base.

8-14. (canceled)

15. A method for treating symptoms from nicotine withdrawal comprising administering to a patient an effective amount of a transdermal composition having the following active ingredients: Sertraline with an active amount of 3% of the transdermal composition, Imipramine with an active amount of 0.5% of the transdermal composition, Topiramate with an active amount of 5% of the transdermal composition, and Bupropion with an active amount of 3% of the transdermal composition.
